(19) United States
(12) Patent Application Publication
(10) Pub. No.: US 2003/0087397 A1
(43) Pub. Date: May 8, 2003

(54) Multiplex real-time PCR

(75) Inventors: Dieter Klein, Tulln (AT); Walter H. Gunzburg, Modling (AT); Brian Salmons, Modling (AT)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Bavarian Nordic Research Institute A/S, Copenhagen V (DK)

(21) Appl. No.: 09/919,501

(22) Filed: Jul. 30, 2001

Related U.S. Application Data
(63) Continuation-in-part of application No. PCT/EP00/00677, filed on Jan. 28, 2000.

(30) Foreign Application Priority Data
Jan. 29, 1999 (DK) PA 1999 00114

(51) Int. Cl. C12Q 1/70; C12Q 1/68; C12P 19/34; C07H 21/04; C12P 21/06

(52) U.S. Cl. 435/91.1; 435/5; 435/6; 435/91.2; 435/69.1; 435/91.33; 536/24.33

(57) ABSTRACT

The present invention relates to a real-time Polymerase Chain Reaction (PCR) method for the detection and quantification of variants of nucleic acid sequences, which differ in the probe-binding site. The method is based on the complete and/or partial amplification of the same region of the variants and the addition of two or more oligonucleotide probes to the same PCR mixture, each probe being specific for the prob-binding site of at least one variant. The method can be applied e.g. to estimate the viral load in a sample, to differentiate between subgroups, subtypes isolates or clades of a viral species or to estimate the impact of the viral load on tumorgenesis.

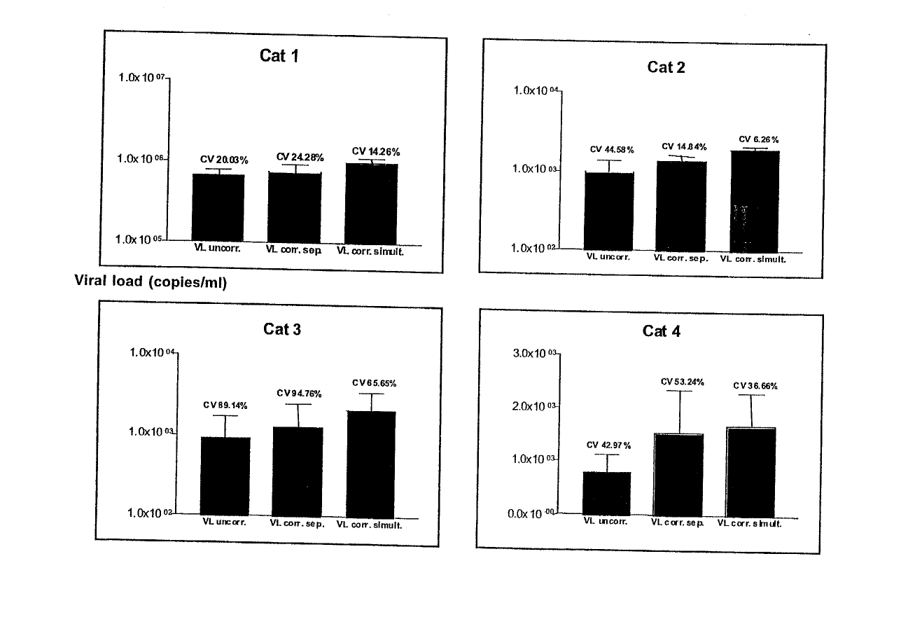

### RELATED APPLICATION(S)

This application is a continuation-in-part of International Application No. PCT/EP00/00677, which designated the United States and was filed on Jan. 28, 2000, published in English, which claims the benefit of Danish Application No. PA 1999 00114, filed on Jan. 29, 1999. The entire teachings of the above application(s) are incorporated herein by reference.

### BACKGROUND OF THE INVENTION

The detection and quantification of nucleic acid sequences is of importance for a wide range of experiments and applications. Several methods for the detection and quantification of nucleic acid sequences have been described previously. Most methods are based on polymerase chain reaction (PCR): The PCR is used to amplify a segment of DNA flanked by stretches of known sequences. Two oligonucleotides binding to these known flanking sequences are used as primers for a series if in vitro reactions that are catalyzed by a DNA polymerase. Typically, these oligonucleotides have different sequences and are complementary to sequences that (1) lie on opposite strands of the template DNA and (2) flank the segment of DNA that is to be amplified. The template DNA is first denatured by heat in the presence of large molar excess of each of the two oligonucleotides and the four dNTPs. The reaction mixture is then cooled to a temperature that allows the oligonucleotide primers to anneal to their target sequences. Afterwards, the annealed primers are extended by the DNA polymerase. The cycle of denaturation, annealing, and DNA-synthesis is then repeated about 10 to 50 times. Since the products of one cycle are used as a template for the next cycle the amount of the amplified DNA fragment is theoretically doubled with each cycle resulting in a PCR-efficiency of 100%.

The specific amplification of a target sequence is due to the annealing of the primers to a complementary region of the DNA. If the primer differs in its sequence from the sequence of the annealing region of the target DNA, the PCR may fail. Accordingly, if a target sequence is analyzed that differs between samples in the primer-annealing region, the amplification of the target sequence in some samples will fail or will be less efficient. Therefore, degenerated primers are often used, i.e. primers that have unspecific nucleotide analogous at the positions at which the sequence varies between samples.

If two or more target sequences are amplified simultaneously in the same PCR reaction, a multiplex PCR is performed. Then, more than one primer pair is added to the PCR mixture and each primer pair allows the specific amplification of one target sequence.

The enzyme used for PCR is specific for DNA. If an RNA template is amplified by PCR, the RNA has first to be transcribed into complementary DNA (cDNA) by the enzyme reverse transcriptase. Afterwards the cDNA is used as a template in a PCR. Accordingly, the method of the amplification of RNA is called reverse-transcriptase (Rt) PCR.

The PCR results in a large copy number of the sequence flanked by the primers. The large copy number of this sequence allows the detection and quantification of the target sequence after the PCR reaction. The detection of the amplification products is usually performed by gel electrophoresis and staining of the DNA. The intensity of the band after gel electrophoresis also allows to estimate the copy number of the sequence of interest in the original sample mostly by comparison with a standard with a known copy number (Sambrook et al., Molecular Cloning, 2^{nd} edition, Cold Spring Harbor Laboratory Press 1989,p. 14.30).

The conventional PCR is widely used. However, the method has several disadvantages that are mostly linked with the detection of the amplification products by gel electrophoresis. The gel electrophoresis requires additional handling of the sample which is time-consuming and prone to sample mix-ups. In addition, the sensitivity of the detection method is low. Finally, quantification of the copy number of the template sequence requires a standard and is often difficult.

More recently, a new technology for the detection and quantification of target sequences was developed which does not show the disadvantages mentioned above. The method is called “real-time” PCR. Here, the DNA generated within a PCR is detected on a cycle-by-cycle basis during the PCR reaction. The amount of DNA increases the faster the more template sequences are present in the original sample. When enough amplification products are made a threshold is reached at which the PCR products are detected. Hence, amplification and detection are preformed simultaneously in the same tube.

Most instruments that are used for a real-time PCR detect an increase of fluorescence of a specific wave length as a result of an increasing amount of PCR product. For example, the Applied BioSystems Prism 7700 sequence detection system is based on the combination of PCR and hybridization of a fluorogenic, target-specific probe. The probe is an oligonucleotide with both a reporter and a quencher dye attached at the 5′ and 3′ end respectively. The fluorescence of the reporter dye is efficiently quenched by the quencher dye as long as both fluorochromes are present in close proximity. If the target sequence is present, the probe anneals between the forward and reverse primers. During PCR amplification and thus elongation of the primers the probe is cleaved by the 5′ nuclease activity of the DNA-polymerase. This cleavage of the probe separates the reporter dye from the quencher dye, making reporter dye signal detectable. Additional reporter dye molecules are cleaved from their respective probes with each cycle, effecting a proportional increase in fluorescence intensity of the reporter dye and a decrease of the fluorescence intensity of the quencher dye. An algorithm of the software of the instrument compares the amount of reporter dye emission with the quenching dye emission once every few second during the PCR reaction, generating a normalized reporter signal ΔRₙ. The first cycle in which the normalized reporter signal is above a defined threshold is defined as the threshold cycle C_{T}. The C_{T} value is proportional to the copy number of the template and used for quantification (Heid, et al., Genome Research, 6: 986ff). The real-time PCR provides greater quantitative precision and dynamic range compared to other quantitative PCR methods, and is easier to handle.

If the template is not DNA but RNA a real-time reverse-transcription (RT) PCR is performed. As described for the conventional PCR the RNA is first transcribed into cDNA before the actual real-time PCR is performed.

### SUMMARY OF THE INVENTION

The present invention relates to:

A real-time Polymerase Chain Reaction (PCR) method for the detection and/or quantification of variants of a nucleic acid sequence, wherein the same region of said variants is completely or partially to be amplified, each variant differing in one or more nucleotides within the probe-binding site, said method comprising addition of two or more oligonucleotide probes to the same PCR mixture, each probe being specific for the probe-binding site of at least one variant.

The real-time PCR method as above, wherein said variants of the nucleic acid sequence differ in one or more nucleotides within the primer-binding sites and wherein more than one primer pair is added to the reaction mixture each primer specifically annealing to the primer-binding site of at least one subtype.

The real-time PCR method as above, wherein two or more parts of the region of each variant are amplified, each part of the region comprising only one probe-binding site.

The real-time PCR method as above, wherein the different probes are labeled with different fluorescent reporter dyes.

The real-time PCR method as above, wherein the probes are labeled with FAM™ or VIC™.

The real-time PCR method as above for the detection and/or quantification of variants of the nucleic acid sequence of a virus.

The real-time PCR method as above for the detection and/or quantification of variants of nucleic acid sequence of a retrovirus.

The real-time PCR method as above for the detection and/or quantification of variants of the nucleic acid sequence of a lentivirus.

The real-time PCR method as above for the detection and/or quantification of variants of the nucleic acid sequence of a Feline Immunodeficiency Virus (FIV).

The real-time PCR method as above, wherein the probes according to Seq. ID No. 3 and 6 are added to the reaction mixture.

The real-time PCR method as above, wherein the probes according to Seq. ID No. 3 and 9 are added to the reaction mixture.

The real-time PCR method as above, wherein the forward primer according to Seq. ID No. 1 and/or 12 and the reverse primer according to Seq. ID No. 2 are added to the reaction mixture.

The real-time PCR method as above, wherein the forward primer according to Seq. ID No. 4, 14, and/or 15 and the reverse primer according to Seq. ID No. 5 and/or 13 are added to the reaction mixture.

The real-time PCR method as above, wherein the forward primer according to Seq. ID No. 7, 20, and/or 21 and the reverse primer according to Seq. ID No. 8 are added to the reaction mixture.

The real-time PCR method as above, wherein the forward primer according to Seq. ID No. 16 and the reverse primer according to Seq. ID No. 17 or 19 are added to the reaction mixture.

The real-time PCR method as above, wherein the above listed primers and probes are added to the reaction mixture.

The real-time PCR method as above, wherein said PCR is a reverse-transcription (RT) PCR.

The real-time PCR method as above, wherein said variants of nucleic acid sequences are nucleic acid sequences derived from subtypes, isolates, clades or any other subgroup of a species.

Use of the real-time PCR method, as above, for the determination of the overall viral load in a sample comprising variants of a viral nucleic acid sequence.

The use of the real-time PCR method, as above, wherein the variants are derived from nucleic acid sequences derived from subtypes, isolates, clades or any other subgroup of a viral species.

The use of the real-time PCR method as above for the investigation of the impact of the viral load on tumorgenesis.

An oligonucleotide probe for use in a real-time PCR method selected from the group of probes comprising:

(a) the nucleic acid sequences according to Seq. ID No. 3 and/or Seq. ID No. 6 and/or Seq. ID No. 9 and/or Seq. ID No. 18,

(b) their complementary strands, and/or

(c) nucleic acid sequences with a homology of at least about 70% to the nucleic acid sequences according to Seq. ID No. 3 and/or Seq. ID No. 6 and/or Seq. ID No. 9 and/or Seq. ID No. 18.

A primer for use in a PCR method selected from the group of primers comprising:

(a) a primer according to Seq. ID No. 1, 2, 4, 5, 7 or 8 or 10 to 17 or 19 to 21,

(b) a primer complementary to one of said sequences, and/or

(c) a primer with a homology of at least about 70% to the nucleic acid sequences of one of said primers.

A set of primers selected from the group of primer sets comprising:

(a) the primers according to Seq. ID No. 1 and/or 12 and according to Seq. ID No. 2,

(b) primers with a nucleic acid sequences complementary to one or more of the primers according to (a), and/or

(c) a primer with a nucleic acid sequence with homology of at least about 70% to the primers according to (a). A set of primers selected from the group of sets of primers comprising:

(a) the primers according to Seq. ID No. 4, 14, and/or 15 and according to Seq. ID No. 5 and/or 13,

(b) primers with a nucleic acid sequence complementary to one or more of the primers according to (a), and/or

(c) a primer with a nucleic acid sequence with homology of at least about 70% to the primers according to (a). A set of primers selected from the group of sets of primers comprising:

(a) the primers according to Seq. ID No. 7, 20, and/or 21 and according to Seq. ID No. 8,

(b) primers with a nucleic acid sequence complementary to one or more of the primers according to (a), and/or

(c) a primer with a nucleic acid sequence with a homology of at least about 70% to the primers according (a). A set of primers selected from the group of sets of primers comprising:

(a) the primers according to Seq. ID 16 and according to Seq. ID No. 17 and/or 19,

(b) primers with a nucleic acid sequence complementary to one or more of the primers according to (a), and/or

(c) a primer with a nucleic acid sequence with a homology of at least about 70% to the primers according to (a).

A set of oligonucleotides for use in a real-time PCR method, comprising a primer set selected from the group of primer sets as above and a probe selected from the group of probes as above.

The set of oligonucleotides as above for use in the method as above.

### BRIEF DESCRIPTION OF THE DRAWING

The FIGURE is four bar graphs showing the influence of extraction efficiency on viral load. Four different plasma samples from FIV infected cats were spiked with 10⁹ copies EGFP RNA, and total RNA were extracted in triplicate. The FIV RNA copy number in each of the triplicate samples was estimated in a multiplex real-time RT-PCR and used to calculate the viral load without compensation of the losses during extraction (VL uncorr.; mean +/−SD). The recovery rate was calculated using a second real-time RT-PCR for the quantification of the EGFP RNA. The calculated recovery rates were used to compensate for the differences in the nucleic acid preparation, and the corrected viral load data are illustrated in the second bar (VL corr. sep.; mean +/−SD). The same uncorrected viral load data were alternatively corrected using the individual recovery rates estimated in a multiplex real-time RT-PCR assay. The results are illustrated in the third bar (VL corr. simult.; mean +/−SD). The corresponding coefficients of variation (CV) are given above each bar.

### DETAILED DESCRIPTION OF THE INVENTION

For the determination of the viral load in samples from animals infected with the same type of virus a real-time PCR was performed. After detection and quantification of the viral nucleic acid sequence by real-time PCR the viral load was calculated. Considerable variation was found between the samples. In some samples the calculated copy number of the target sequence was very small or even no target sequence was detected. Unexpectedly, the calculated viral load did not correlate with the severity of the disease of those animals the samples were taken from. In order to verify the results of the real-time PCR, a conventional PCR with staining of the amplified DNA after gel electrophoresis was performed. Although this method is much less sensitive as compared to the real-time PCR method, amplification products were surprisingly detected in all analyzed samples, i.e. even for samples in which no viral sequences were detected by real-time PCR, positive results were obtained by conventional PCR.

Further investigations showed that the animals the samples were taken from were infected with different subtypes of the same virus. The subtypes were characterized by variants of the viral nucleic acid sequence. Apparently, some variants of a nucleic acid sequence that were present in specific viral subtypes, were not detected by real-time PCR.

Accordingly, it was an object of the present invention, to provide a real-time PCR method for the detection and/or quantification of variants of a nucleic acid sequence.

The problem underlying the present invention is solved by the complete and/or partial amplification of the same region of variants of a nucleic acid sequence comprising nucleotide variations within the probe-binding site and the addition of two or more oligonucleotide probes to the same PCR mixture, each probe being specific for the probe-binding site of at least one variant. Said variants of a nucleic acid sequence are found e.g. in different subtypes of phylogenetically related groups of organisms such as in subtypes of families, genera, and species. The variants analyzed by a method according to the present invention are preferably derived from subtypes of a species such as e.g. clades, isolates or breeds. The variants of nucleic acid sequences may be identical in about 50 to about 70%, preferably about 70 to about 90% and most preferably about 90 to about 99% of the nucleotides.

The nucleic acid sequence of the different subtypes may differ not only in the probe-binding site but also in the primer-binding site. In this case, primers may not anneal to the primer-binding site, resulting in PCR failure. Hence, according to a preferred embodiment of the invention, more than one primer pair is added to the reaction mixture, wherein each primer specifically anneals to the nucleic acid sequence of at least one subtype (multiplex real-time PCR).

The primers and probes used for the method according to the present invention should be at least about 60 to about 80%, preferably about 80 to about 90%, and most preferably about 90 to about 100% homologous to the nucleic acid sequence of at least one variant of the nucleic acid sequence.

Within the completely or partially amplified region more than one probe-binding site may be included. If in this case the complete region is amplified the amplification products have more than one probe-binding site and more than one probe may anneal to the amplification product. This may e.g. cause interactions between the reporter and quencher dyes of the different annealed probes influencing the quantification. Hence, according to a preferred embodiment of the invention, two or more parts of the region of each variant are amplified, each part of the region comprising only one probe-binding site.

When two or more parts of the region of the nucleic acid sequence are amplified, primer pairs and probes may be chosen to be specific for one variant. In this case, the fluorescence signal of a specific probe may be characteristic for a specific variant.

The probes are labeled at the 3-prime end with a quencher and at the 5-prime end with a reporter dye. According to a preferred embodiment of the invention, different probes are labeled with the same quencher dye but with different reporter dyes. In that case, the different amplification products can be distinguished. Any reporter dye can be attached to the probe. However, preferably FAM™ or VIC™ is used as a reporter dye.

The differentiation between amplification products using different reporter dyes may be applied for the classification of subtypes. First the inventors classified the subtypes by monoplex real-time PCRs. In this case, only one primer pair and one probe is added to the reaction mixture, wherein the primer-pair and probe are chosen to be specific for one subtype. Then, PCR products should only be detected if this specific subtype is present in the sample. However, non-specific amplification and/or detection was observed in some cases, resulting in wrong classification of the subtype. Then the inventors used a multiplex real-time PCR according to the present invention for the classification. They added several of the subtype-specific primers and probes to a PCR mixture, wherein the different probes were labeled with different reporter dyes. In this case, a specific subtype is identified, when the fluorescence signal of the respective probe is detected. Using this multiplex real-time PCR method, all subtypes could be classified correctly. Hence, according to a preferred embodiment of the present invention, a multiplex real-time PCR with subtype-specific primers and probes is performed for the classification of subtypes, wherein the probes are labeled with different reporter dyes.

The present invention may be used to study viral diseases such as diseases caused by lentiviruses. Lentiviruses are associated with immunodeficiency and malignancies. The mechanisms involved in tumorgenesis are still not fully understood, but it is suspected that a correlation between tumorgenesis and the viral load exists. Cats infected with Feline Immunodeficiency Virus (FIV) represent a model for the role of the viral load in the pathogenesis of tumors, since cats infected with FIV develop quite often tumors, especially lymphomas. Accordingly, in a preferred embodiment of the present invention, the real-time PCR is especially used for the detection and/or quantification of nucleic acid sequences of different subtypes of lentiviruses, especially of FIV.

Since lentiviruses are retroviruses, the nucleic acid sequence of the genome present in the viral particles consists of RNA. According to the life cycle of a retrovirus, the RNA genome is transcribed into DNA after the infection of a host cell. Then, the transcribed, retroviral DNA may be integrated into the genome of the host cell, forming the so-called provirus. If the already integrated viral genome shall be analyzed and accordingly amplified, no reverse transcription is necessary before the real-time PCR is performed. However, if the genome of the viral particles is analyzed, a reverse-transcription (RT) real-time PCR is performed.

FIV isolates comprising known and unknown variants of the viral nucleic acid sequence have been analyzed by FIV specific real-time PCR. Accordingly, the present invention also provides oligonucleotide probes as well as primer pairs for the detection and/or quantification of variants of nucleic acid sequences derived from FIV. Preferably probes and primer pairs according to Seq. ID No. 1 to 24 are provided. The primers and probe according to Seq. ID No. 1 to 3 are especially used for the detection and/or quantification of lade A of FIV, whereas oligonucleotides according to Seq. ID No. 7 to 9 are specifically used for the detection and/or quantification of clade B of FIV. Preferable, both sets of oligonucleotides are used simultaneously in a multiplex real-time PCR. Accordingly, with the two sets of oligonucleotides a method is provided which enables to distinguish between dade A and B of FIV in a sample. The probes according to Seq. ID No. 3, 6, 9, 18 or 24 may be combined with primers different from those according to Seq. ID No. 1, 2, 4, 5, 7 or 8, especially when FIV samples are analyzed that comprise unknown FIV isolates. In one embodiment of the invention, the forward primer according to Seq. ID No. 1 is replaced by a primer according to Seq. ID No. 12. The probe according to Seq. ID No. 6 may be used in combination with the forward primers according to Seq. ID No. 4, 14 or 15 and the reverse primers according to Seq. ID No 5 or 13. The probe according to Seq. ID No. 9 maybe used in combination with the forward primer according to Seq. ID No. 7, 20 or 21 and the reverse primer according to Seq. ID No. 8. The probe according to Seq. ID No. 18 may be used in combination with the forward primers according to Seq. ID No. 1, 10, 12 or 16 and the reverse primers according to Seq. ID No. 5, 8, 11, 13, 17 or 19. The probe according to Seq. ID No. 24 may be used in combination with the forward primers according to Seq. ID No. 10 or 22 and the reverse primers according to Seq. ID No. 2, 5, 8, 11, 13, 17, 19 or 23. Furthermore, a set of oligonucleotides is provided comprising the probe according to Seq. ID No. 18, the forward primer according to Seq. ID No. 16, and the reverse primer according to Seq. ID No. 17 or 19.

The primers and probes with a sequence according to Seq. ID No. 1 to 24 as well as primers and probes with a homology of at least about 70% to the sequences according to Seq. ID No. 1 to 24 may be used in general to amplify sequences specific for FIV. Additionally, the primer pairs can also be applied without the probes when a conventional PCR is performed instead of a real-time PCR.

In summary, the present invention provides a highly reliable and reproducible method for the detection and quantification of variables of nucleic acid sequences.

As a further aspect of the present invention as consequence of this improved precision in the quantitative RT-PCR pre-assay variations must be considered. Particularly, pre-assay variations due to nucleic acid preparations and storage have greater impact on the accuracy of the viral load determination.

The overall precision of viral load quantification in serum or plasma samples is not only dependent on a precise quantification method, but also on the reproducibility of the pre-analytical stages, i.e. minimal and reproducible losses during storage and RNA preparation from the sample. To determine the influence of pre-assay variations on viral load estimation a known amount (10⁹ copies) of enhanced green fluorescent protein (EGFP) RNA was used to spike four different FIV samples. The RNA of these spiked samples was extracted under identical conditions. The EGFP copy number after extraction was estimated with a second optimized real-time PCR system for the EGFP gene and was the basis for the calculation of the recovery rate. The calculated recovery rates of the four spiked samples (each prepared in triplicate) ranged from 23% to 64%. These recovery rates were used to compensate for the individual loses during nucleic acid preparation. The compensation of the individual loses during nucleic acid extraction should increase the accuracy of the viral load measurement. However, in order to check if this compensation has increased as well, the precision of the viral load measurement the coefficient of variation (CV) of the calculated copy numbers from the triplicate measurements of each sample before and after correction with the compensation factor was compared. Interestingly, only in one of the four samples was the CV of the copy number between the triplicate preparations lower after correction, and the overall calculated mean CV of the four samples increased from 26,12% to 33,96%. One possible explanation for this unexpected lower precision of the viral load measurements is that two independent real-time RT-PCRs are carried out for each sample, one to estimate the FIV copy number and one for the correction factor. A cumulation of the errors in each RT-PCR might explain this lower precision. To circumvent this problem a multiplex real-time RT-PCR, which allows the estimation of the FIV copy number and the correction factor simultaneously in one tube, was used. This multiplex real-time RT-PCR allowed a rapid and accurate calculation of copy number independent of pre-assay variations.

Another goal is a system that allows the detection and/or quantification of a broad range of different isolates with similar PCR efficiencies. Based on the optimized system according to the present invention the comparison of viral loads e.g. from cats infected with different FIV isolates can be performed much more precisely. Such an optimized system provides the tool for the investigation of the impact of the viral load on the development of cancers in lentiviral infection, was well as provided the basis for the investigation of the efficiency of therapeutic agents tested in naturally infected cats. Furthermore, real-time PCR strategies can be designed to detect mutations in oncogenes present in biopsy material, where tumor and normal cells may be present. In such cases, real-time PCR permits a quantification of the number of tumor cells present.

### EXAMPLES

The following example will further illustrate the present invention. It will be well understood by a person skilled in the art that the provided example in no way may be interpreted in a way that limits the applicability of the technology provided by the present invention to this example.

### Detection of Proviral DNA

Recently, a method based on the ABI 7700 system (Perkin Elmer, Foster City, Calif.) was established and validated for the quantification of FIV proviral and viral loads (Leutenegger et al. J. Virol Methods 1999, 78(1):105-116). In this method, the 5′ nuclease activity of the Taq-polymerase allows the cleavage of a labeled probe and the subsequent liberation of a reporter fluorescent dye which can be excited with an argon laser and leads to the emission of light. The amount of emitted fluorescence, which is proportional to the amount of DNA produced during the PCR, is measured at regular intervals during the PCR and allows the monitoring of the PCR in a real-time manner. The method has been shown to be very useful in viral load determinations if just one isolate is used (e.g. in challenge experiments for vaccination trials where the isolate is known and optimized primers and probes could be used). However, as soon as viral loads from different isolates should be compared the equality of the PCR efficiency for the different isolates must be ensured. In the following example, the influence of mutations in the primer- and probe-binding site on the PCR-efficiency and the subsequent estimation of this influence on the viral load determination is analyzed. A real-time PCR system was established that allows the estimation of viral loads in patients infected with different isolates, as a basis for the determination of the impact of the viral load on tumorgenesis.

### Conditions and Parameters of the Real-time PCR for the Analysis of FIV Sequences

First the conditions for the amplification of FIV sequences by real-time PCR were established and evaluated. Primers and probes for the amplification and detection of the gag gene of FIV were designed. Then the linear range within which the copy number of a template can be quantified was calculated using a plasmid with a corresponding region of a FIV isolate. Additionally, PCR-efficiencies were calculated for this plasmid and for a genomic standard.

### Real-time PCR Primers and Probes

For the real-time PCR the subsequently described primer and probe sequences were designed using the Primer Express software (Perkin Elmer, Foster City, Calif.). All oligonucleotides were purified by high-performance liquid chromatography and purchased from Perkin Elmer (Weiterstadt, Germany).

Several different real-time PCR assays (FIV1010p/v, FIV581p, FIV1416p, FIV1212v, FIV1372p, and EGFP234p) were developed.

For example the FIV1010p-assay uses the PCR primers FIV0771f (5′- AGA ACC TGG TGA TAT ACC AGA GAC - 3′) (SEQ ID NO.:1) and FIV1081r (5′- TTG GGT CAA GTG CTA CAT ATT G - 3′) (SEQ ID NO.:2). The primers were designed to be 100% homologous to the sequence of the clade A FIV isolates, which comprise among other strains the isolates Petaluma (Genebank accession number M25381), San Diego PPR (M36968), Zurichl (X57002), and Utrechtl 13 (X68019). It was also considered that for an efficient amplification the size of the amplified fragment should be smaller than about 350 bp and, if possible, smaller than about 100 bp.

Regarding the used reporter dyes for a monoplex PCR, the probe was labeled at the 5′ end with the fluorochrome FAM (6-carboxy-fluorescein), which serves as a reporter fluorochrome and at the 3′ end with the fluorochrome TAMRA (6-carboxy-tetramethyl-rhodamine) which functions as a quencher. In a multiplex PCR the same probe was labeled with the reporter fluorochrome VIC to distinguish between the signal of the different PCR systems.

The used probes were designed, based on several criteria: (i) 8-10° C. higher melting temperature than the primers, (ii) no G's at the 5′ end of the probe, (iii) no stretches of identical nucleotides longer than four, especially not of G's, (iv) lack of self-annealing, (v) lack of predicted dimer formation with corresponding primers.

Furthermore, the probe is blocked at the 3′ end to prevent elongation during the amplification. The probe was at least about 80% but preferably about 95% to about 100% homologous to the sequence of different FIV isolates. The probe used to establish standard assay conditions was FIV1010p/v (5′-FAM/VIC-TAT GCC TGT GGA GGG CCT TCC T-TAMRA-3′) (SEQ ID NO.:3).

Under the same considerations as described for the FIV 1010p-assay the other assays were designed.

The second FIV assay (FIV581p) was designed to be 100% homologous to the clades A FIV isolates Petaluma and Zurich 2 (FIV Z2) and consisted of the primers FIV551f (5′-GCC TTC TCT GCA AAT TTA ACA CCT-3′) (SEQ ID NO.:22) and FIV671r (5′-GAT CAT ATT CTG CTG TCA ATT GCT TT-3′) (SEQ ID NO.:23) and the probe FIV581p (5′-FAM-TGC GGC CAT TAT TAA TGT GGC CAT G-TAMRA-3′) (SEQ ID NO.:24). Both FIV systems have been previously shown to detect in separate setups FIV isolates from clade A and B.

A further FIV assay (1416p) was designed to detect a broad range of Austrian and German FIV isolates and consists of the probe FIV1416p (5′-FAM-TGC AGT GTA GAG CAT GGT ATC TTG AGG CA-TAMRA-3′). The probe can be combined with two different forward primers (FIV1360f: 5′-GCA GAA GCA AGA TTT GCA CCA-3′ (SEQ ID NO.:4) and FIV1366fa: 5′-GCA AGA TTT GCA CCA GCT AGG-3″ (SEQ ID NO.:14)) and two different reverse primers (FIV1437r: 5′-TAT GGC GGC CAA TTT TCC T-3′ (SEQ ID NO.:5) and FIV1437rb: 5′-TAT GGC TGC CAA CTT TCC T-3′ (SEQ ID NO.:13)).

The probe used for the fourth FIV assay was labeled with a different reporter fluorochrome (VIC) to enable the setup of a multiplex assay for the detection of local FIV isolates and consists of the probe FIV1212v (5′-VIC-TGC-GCT GCA GAT AAA GAA ATA TTG GAT GA-TAMRA-3′), the forward primer FIV1182f (5′-ATG GCC ACA TTA ATA ATG GC-3′) (SEQ ID NO.:16), which can combined with two different reverse primers (FIV1307r: 5′-GGT AAT GGT CTA GGA CCA TCA-3′ (SEQ ID NO.:17) and FIV1307z: 5′-GGT AAT GGT CTG GGA GCA TCA-3′(SEQ ID NO.:19)).

The FIV1372p-assay was designed to be 100% homologous to clade B FIV isolates Italy-M2 (Y13867), Italy-M3 (Y13866), Italy-M8 (Z961 11), Amori-1 (D37823), Amori-2 (D37824), Sedai-2 (D37821), Yokohama (D37819), and a local, subtype B-like isolate. The system consisted of the primers FIV1280f (5′-ATC CTC CTG ATG GGC CTA GAC-3′) (SEQ ID NO.:7) and FIV1426r (5′-ACT TTC CTA ATG CTT CAA GGT ACC A-3′) (SEQ ID NO.:8) and the probe FIV1372p (5′-TTT GCA CCA GCC AGA ATG CAG TGT AG-3′) (SEQ ID NO.:9).

The probe of the EGFP assay system (EGFP234v; 5′-VIC-CCG ACC ACA TGA AGC AGC ACG ACT T-3′-TAMRA) (SEQ ID NO.:27) was as well labeled with a different reporter fluorochrome (VIC) to enable the setup of an optimal FIV/EGFP multiplex RT-PCR. The primers used together with the probe were EGFP214f (5′-GCA GTG CTT CAG CCG CTA C-3′) (SEQ ID NO.:25) and EFP309r (5′-AAG AAG ATG GTG CGC TCC TG-3′) (SEQ ID NO.:26).

All five probes are phosphorylated at the 3′-OH to prevent elongation during the PCR. All oligonucleotides were high-performance liquid chromatography purified and obtained from Perkin-Elmer (Weiterstadt, Germany).

### Cycling Conditions for PCR

The target sequence was amplified in a 25 μl reaction volume using the following PCR-conditions: 10 mM Tris (pH 8.3), 50 mM KC1, 3 mM MgCl₂, 200 nM dATP, dCTP, dGTP, 400 nM dUTP, 300 nM of each primer, 200 nM of the fluorogenic probe, and 2.5 units of Taq DNA polymerase were used. After the initial denaturation (2 min at 95° C.), amplification was performed in 45 cycles each comprising 15 sec at 95° C. and 60 sec at 60° C. For a multiplex PCR the same reaction conditions were applied. In case a reverse transcription PCR was used the following protocol was used.

The 50 μl RT-PCR mixtures contained 10 μl AMV/Tfl5x reaction buffer (Access RT-PCR system, Promega, Mannheim, Germany), 3 mM MgSO₄, 200 μM dATP, dCTP, dGTP, dTTP, 300 nM of each primer, 200 nM of the fluorogenic probe, 5 U of AMV reverse transcriptase, 5 U of Tfl DNA polymerase and 5 μl of the sample or RNA standard. After a reverse transcription step of 45 min. at 48° C. followed by a denaturation step (2 min. at 95° C.), amplification was performed with 45 cycles of 15 sec at 95° C. and 60 sec at 60° C.

For multiplex RT-PCR the same reaction conditions were used, but the primer concentration of the abundant system (EGFP234v) were limited to allow efficient amplification of the rarer target RNA (FIV). However, they have still to be high enough to ensure efficient quantification of the EGFP RNA. A concentration of about 100 nM of the EGFP primers was found to be optimal for these purposes.

Real-time RT-PCRs used for the estimation of reaction efficiencies were performed using a total volume of 25 μl containing 12,5 μl 2×Thermoscript Reaction Mix (Platinum Quantitative RT-PCR Kit, Life Technologies, Karlsruhe, Germany), 300 nM of each primer, 200 nM of the fluorogenic probe, 0,5 μl of the Thermoscript Plus/Platinum Taq Enzyme mix, 20 Uof RnaseOUT (Life Technologies, Karlsruhe, Germany) and 5 μl of the sample. After a reverse transcription step of 30 min. at 60° C. followed by a denaturation step (5 min. at 95° C.), amplification was performed with 45 cycles of 15 sec at 95° C. and 60 sec at 60° C.

Reverse transcription and amplification were performed in an ABI Prism® 7700 Sequence Detection System (Perkin-Elmer, Foster City, Calif.). The detected fluorescence signals are analyzed using the Sequence Detection Software Version 1.6.3 (Perkin-Elmer, Foster City, Calif.).

### Sample Preparation

### Preparation of Standard DNA Templates

A Construct of the FIV Zrish 2 isolate (plasmid, pBSCompZ2 [Allenspach, et al., Schweiz Arch Tierheilkd 1996, 138, 87-92]) was used as a control to determine the linear range of the real-time PCR. The plasmid was propagated in E. coli cells and extracted using the Qiagen Plasmid Kit according to manufacturer's instructions (Qiagen, Hilden, Germany). The copy number of the plasmid was estimated from the absorption at 260 nM. A set of tenfold dilutions was performed in PCR grade water containing calf thymus DNA as a carrier in a concentration of 30 μg/ml.

A genomic DNA standard was developed that mimics the in vivo situation of a provirus integrated into genomic DNA of a cell. The DNA from CrFK cells [Crandell, et al., In Vitro 1973, 9, 176-185] stably infected with different FIV isolates (Petaluma [Pedersen, et al., Science 1987, 235, 790-793], Glasgow 8 [Hosie & Jarrett, Aids 1990, 4, 215-220], Amsterdam 6 [Siebeline, et al., Vet Immunol Immunopathos 1995, 46, 61-69], Utrecht 113 [Verschoor, et al., J Clin Microbiol 1993, 31, 2350-2355] was extracted using the QIAamp Kit according to manufacturer's instructions (Qiagen, Hilden Germany). The DNA concentration was estimated by OD measurement at 260 nm and a tenfold dilution series was performed in PCR grade water containing 30 μg cellular DNA per ml.

When the proviral load was studied DNA was extracted from peripheral blood leucocytes. Otherwise, it was proceeded as described above for the DNA-extraction from infected CrFK cells.

### Preparation of Standard RNA Templates for RT-PCR

The plasmid pBSCompZ2 [Allenspach K, et al. Schweiz Arch Tierheilkd 1996; 138:87-92] containing the corresponding region of the FIV Zurich 2 isolate was digested with BamHI. The linearized plasmid was used as template for in vitro transcription using the RiboProbe kit (Promega, Mannheim, Germany) according to the manufacturer's instructions. The FIV RNA copy number was calculated after OD measurement at 260 nm. Tenfold dilution series were performed in nuclease free water containing 30 μg tRNA (Boehringer, Mannheim, Germany) per ml as carrier RNA.

### Sample Preparation

Plasma samples of FIV infected cats and supernatants of persistently FIV infected cell lines were extracted using the QIAamp Viral RNA Kit (Qiagen, Hilden, Germany) according to the manufacturer's instructions. In order to determine the extraction efficiency 10⁹ EGFP RNA molecules per extraction were added to the lysis buffer of the kit. The samples used for the comparison of the reaction efficiencies were diluted in fourfold serial steps in nuclease free water containing 3 μg t-RNA per ml.

### Calculation of Copy Number

An algorithm of the Sequence Detection Software compares the amount of reporter dye emission (R) with the quenching dye emission (Q) once every few seconds during the PCR amplification, generating a normalized reporter signal ΔRₙ. This value represents the fluorescence signal of the reporter dye divided by the fluorescence signal of the quencher signal minus the baseline signal established in the first few cycles of the PCR when cleaved probe is generally not detectable. The ΔRₙ values are plotted as a function of the PCR cycle. The first cycle which is above a defined threshold (normally ten times the standard deviation of the background fluorescence) is defined as the threshold cycle C_{T}. Within a certain range of template concentrations the C_{T} value is proportional to the template copy number present at the beginning of the reaction and reflects the first opportunity for quantification of the template (Heid, et al., 1996, Genome Research, 6, p.986ff).

### Quantification of Control DNA

A dilution of the plasmid pBSCompZ2 was obtained as described above and the concentration was estimated in three independent real-time PCRs using the FIV1010p-assay for each of the diluted samples. The calculation of the initial copy number using ΔRₙ is highly reproducible as it was shown by small standard deviations of the C_{T} values, ranging from 0.13 to 2.99%. With a decreasing number of template copies the number of cycles increased and larger standard deviations are obtained. Hence, the accuracy of the measurement deteriorates.

Using the PCR conditions described above a linear relationship between C_{T} and the standard template concentration was achieved for nine log units (5×10⁰ to 5×10⁹ copies). The coefficient of correlation, that is defined as the percentage of standard deviation of the threshold cycle numbers, was 0.9977. This high coefficient of correlation is a prerequisite for the calculation of the PCR-efficiency. The result confirms that the method is highly accurate over a wide range of template concentrations.

### Calculation of the PCR-efficiency

The PCR-efficiency can be used to evaluate the PCR-conditions. If the PCR-efficiency was 100% the concentration of the target sequence should be doubled every cycle. However, usually the PCR-efficiency (E) is less than 100% and the amount of PCR-product (Y) amplified from an initial template copy number Z after n cycles can be calculated according to the following equation Y=Z×(1+E)ⁿ or after logarthimic transformation as log Y=log Z+n×log (1+E). The PCR-efficiency E can be calculated from the slope of a standard curve where the C_{T}-value is plotted against the logarithm of the copy number of a dilution series. Now the PCR-efficiency can be described as E=10^{-1/s}−1 with s representing the slope of the straight line.

A set of tenfold dilutions of the FIV plasmid was prepared as described above. A real-time PCR using the FIV1010p-assay was performed for each dilution and a standard curve was obtained. A PCR-efficiency of 0.9815 was calculated from the slope of that standard curve.

In addition, the PCR-efficiency of the genomic standard obtained from transduced CrFK cells as described above was calculated. A real-time PCR was performed and PCR-efficiencies were calculated. The PCR-efficiencies varied between 0.9742 and 0.8711. The best PCR-efficiency (0.9742 for CrFK cells infected with FIV Petaluma) is almost as good as the PCR-efficiency of the plasmid dilution series.

For all constructs the correlation coefficient was larger than 0.99 which demonstrates that a comparison of the PCR-efficiencies is possible. The results show that the chosen PCR conditions are suitable for an efficient amplification of the FIV fragment.

### Sequence Analysis of a Conserved Region of the FIV Genome

The complete gag gene (1.6 kb) of the characterized isolates of FIV (Petaluma, Glasgow 8, Amsterdam 6 and Utrecht 113) were amplified and sequenced using the primers FIV566f(5′- ACC TTC AAG CCA GGA GAT TC- 3′) (SEQ ID NO.:10) and FIV2167r (5′-CCT CCT CCT ACT CCA ATC AT-3′) (SEQ ID NO.:11). Additionally, a 311 bp region if the FIVgag gene of some of the unclassified isolates (Munich 3, 4, 6 and 7) was amplified and sequenced with the same primers as for the FIV10101p-assay. The used primers were FIV0771f (5′-AGA ACC TGG TGA TAT ACC AGA GAC-3′) (SEQ ID NO.:1) and FIV1081r (5′-TTG GGT CAA GTG CTA CAT ATT G-3′) (SEQ ID NO.:2).

A conventional PCR was performed on a 9600 thermal cycler (Perkin Elmer, Foster City, Calif.). PCR reactions contained 10 mM Tris (pH 8.3), 50 mM KCl, 3 mM MgCl₂, 200 mM dATP, dCTP, dGTP, dTTP, 300 nM of each primer and 2.5 U of Taq DNA polymerase. Amplification was performed with 1 cycle of 3 min at 95° C., 60 sec at 51° C. and 3 min at 72° C., followed by 39 cycles of 15 sec at 94° C., 40 sec a and 90 sec at 72° C. PCR-products were separated on a 0.8% agarose gel and visualized after ethidium bromide staining with the Eagle Eye system (Stratagene, Heidelberg, Germany). The appropriate bands were isolated and DNA was purified using the QLAmp gel extraction kit (Qiagen, Hilden, Germany) according to the manufacturer's instructions. Approximately 20-50 ng PCR-product were used in the subsequent sequencing reaction mixture containing 4 μl BiDye premix (Perkin Elmer, Foster City, Calif.), 4 pmol of the primer FIV0771f, and water in a total volume of 10 μl. The cycling was performed on a 9600 thermocycler (Perkin Elmer, Foster City, Calif.) with the following program: 30 sec at 96° C., 10 sec at 50° C., 4 min at 60° C. for 30 cycles. The sequencing reaction was purified according to the manufacturer's instructions. The sequence analysis was performed with an ABI 310 Genetic Analyzer (Perkin Elmer, Foster City, Calif.).

### Comparison of Sequence Data and Real-time PCR-efficiency for Standard Isolates

Four isolates were sequenced and, in parallel, amplified by real-time PCR using the FIV100p-assay as described above. The sequence data and the corresponding PCR-efficiencies are listed in Table 1. In the region of the forward primer (FIV077 1 f) no mutation was found in any of the four isolates. In the region where the probe FIV1010p anneals only one mutation was found. The T to C change at bp 1020 was found when the sequence of the Amsterdam 6 isolate [Siebelink, et al., Vet Immunol Immunopathol 1995, 46, 61-69] was compared with the sequence of FIV Petaluma (Genebank accession number M25381). Interestingly, the nucleotide sequence of this isolate was still detected but with the lowest PCR-efficiency (0,8711) compared to all other isolates. In the region of the reverse primer FIV1081r one point mutation was found. In the Glasgow 8 isolate [Hosie & Jarett, Aids 1990, 4, 215-220] and A to T change at position 1071 was detected, which was associated with the second lowest PCR-efficiency of 0.9284. In summary, in the four isolates two point mutations were found, which reduced the PCR-efficiency.

### Comparison of Sequence Data and PCR-efficiencies for Field Isolates

The real-time PCR system was analyzed for FIV isolates of ten naturally infected cats. The cats were selected from southern Germany and Austria. This region has previously been shown to contain a heterogenous FIV population. In that region isolates from three different subtypes and from several other genetic outliers have been found [Bachman, M. H. et al, J. Virol 1997, 71, pp. 4241ff].

Five out of ten tested cats infected with unknown FIV isolates were positive in the real-time PCR assay (Table 2). However, two of the five cats (Munich 3 and 4) that were negative in the real-time PCR, were positive when the PCR-product was analyzed by agarose gel electrophoresis. Sequencing showed that the sequence of the two FIV isolates Munich 3 and 4 differed from the previously published sequences by three and four mutations in the probe-binding site respectively. The mismatches are located in a part of the probe which is initially not displaced by the Taq polymerase and which is responsible for the binding of the probe before the probe is cleaved by the 5′nuclease activity of the Taq-polymerase. The failed amplification can either be explained by the lack of binding or by the displacement of the probe before appropriate cleavage.

In contrast, no mutations were found in the probe-binding site of the two isolates, which were positive in the conventional and in the real-time PCR (Table 2). For these two samples the curve of the real-time showed an exponential increase of the fluorescence signal similar to the one which was seen for the plasmid standard indicating a high PCR-efficiency. In conclusion, variation in the probe-binding site result in apparently reduced PCR-efficiency or even PCR-failure.

### Detection of Different FIV Clades by Multiplex Real-time PCR

The proviral load was studied according to the invention for samples of cats infected with FIV. It was shown that using more than one primer pair and probe allows the detection of a larger number of viral strains and to differentiate between subtypes.

FIV isolates of an unknown subtype were analyzed using the above-described conditions. Three monoplex real-time PCRs were performed: The assays FIV1010v and FIV1416p that are specific for clade A FIV isolates and the FIV1372p-assay that is specific for the lade B subtype. Two multiplex PCRs were performed: the FIV1010v - and the FIV1416p-assay were used in a multiplex real-time PCR to detect dade A isolates. The FIV1010v—and the FIV1372p-assay were used to detect lade A and clade B FIV isolates in a multiplex setup. The use of different reporter dyes (VIC™ in the FIV1010v -assay and FAM™ in the FIV1372p and in the FIV1416p-assay) allowed to distinguish between the signal of the two PCR-systems in the multiplex setup.

The results of the FIV1010p, FIV1416p or the FIV1372p monoplex real-time PCR are compared with the multiplex real-time PCR FIV1010v/FIV1416p and FIV1010v/FIV1372p. The results of the FIV1010p and FV1372p assay are summarized in Table 3. For more than one third of the samples that were analyzed by one monoplex real-time PCR no signal was detected. Surprisingly, combining the FIV1010v and the FIV1372p assaynone of the 30 samples was negative. Hence, the use of more than one primer pair and more than one probe enabled the detection of all samples which were not detected by one monoplex real-time PCR.

The inventors used the results to group the viruses of the different samples into clades. In 6 out of 30 samples the results of the two monoplex real-time PCRs were inconclusive. In contrast, the results of one multiplex real-time PCR allowed the grouping into clade A or B for all samples. In conclusion, the present invention allows to detect sequences of different viral clades and also to group the viruses according to their sequence into the different clades.

### Optimization of the Multiplex Real-time PCR

As mentioned above, in the multiplex PCR, two PCR assays are performed simultaneously in one tube, and thus, share and compete for common reagents, e.g. nucleotides and enzymes. If the two target sequences are not present in similar initial copy numbers, there is a slight possibility for the more abundant target sequence to compete out these common reagents, impairing amplification of the rarer sequence. This situation should be avoided, e.g. by limiting the primer concentration of the detection system for the more abundant target sequence, which than reaches the end-plateau before the shared components are used up. This is especially important in the experiments presented here in which plasma samples are spiked with high amounts of EGFP RNA prior to extraction. These high amounts ensure that the EGFP system is always the more abundant system, but risks the non-detection of low levels of FIV template.

To estimate to which extent the concentration of the primers for the more abundant EGFP target sequence can be reduced without affecting the quantification, but at the same time reducing the number of amplification cycles before the end-plateau value (Rₙ) is reached, a matrix of reactions, each with different concentrations of the EGFP forward and reverse primer was performed in duplicate. The C_{T} values are similar in the range from 100 nM down to 60 nM for both primers. Below a primer concentration of 60 nM the C_{T} value starts to increase, while the Rₙ. value is much lower at 100 and 60 nM compared to 300 nM. In the view of this data, we decided to decrease the primer concentration from 300 nM to 110 nM for both EGFP primers to limit the amplification reaction for this system.

To investigate whether the decreased primer concentration of the EGFP system prevents the impairment of amplification of low levels of FIV template we used a fourfold dilution series of genomic DNA from a persistently FIV infected CrFK cell line (kindly provided by M. Hosie, Glasgow) and mixed it with genomic DNA from a 100% positive EGFP cell line. This standard series thus contains a decreasing amount of FIV template on a background of equal amounts of EGFP template. The standard series was measured in a multiplex real-time PCR either with the normal ratio of the two primer systems (300 nM FIV/300 nM EGFP) or with the limited EGFP primer concentration (300 nM FIV/100 nM EGFP). As expected, the decreased EGFP primer concentration reduces the Rₙ level of the EGFP reporter signal VIC. High FIV copy numbers (26313 copies) are detected in both systems, while low copy numbers (26 copies) are detected only in the limited EGFP primer concentration PCR. This result demonstrates that the decreased EGFP primer concentration of 100 nM is necessary and sufficient to allow the quantification of low FIV copy numbers in this multiplex reaction.

To investigate if the multiplex reaction with the chosen primer ratio of 300 nM to 100 nM (FIV to EGFP) allows efficient quantification of both systems, the two obtained standard curves were compared. Both standard curves displayed high coefficients of correlation (r²>0.998) over 6 logarithmic decades with nearly similar slopes, thus reflecting similar reaction efficiencies for both quantification systems in the multiplex set-up.

This optimized multiplex set-up was then used to estimate the FIV and EGFP copy numbers of the same spiked FIV samples from Table 2, but this time simultaneously in one real-time RT-PCR. The recovery rate estimated from the EGFP copy number in the multiplex system was nearly identical to the results obtained from the monoplex RT-PCR, ranging from 24% to 77%. Again, we checked the precision of the method by comparison of the Cvs before and after correction of the data. In contrast to the data obtained from the two monoplex real-time RT-PCRs, we observed in three of the samples a reduction of the CV of the calculated copy number. The mean CV of the triplicate measurements of the four samples in the corrected multiplex RT-PCR data was much lower (11,94%) than in the uncorrected viral load data (21,48%) indicating the increased precision of viral load estimations estimated with the multiplex RT-PCR approach.

### Influence of Extraction Efficiency on Viral Load

This optimized multiplex real-time RT-PCR assay was then used to determine the influence of the extraction efficiency on the accuracy of the viral load measurement. For this purpose four different plasma samples from FIV infected cats were extracted independently three times using the EGFP RNA spiked lysis buffer. The extracted sample was then used to determine the FIV copy number and the EGRP copy number in a multiplex real-time RT-PCR. Additionally, the EGFP copy number was determined in a separate RT-PCR. Both EGFP copy numbers were independently used to compensate for losses during nucleic acid extraction. The results of all three viral load estimations (viral load uncorrected (VL uncorr.), viral load corrected with a separate RT-PCR (VL corr. sep.) and viral load corrected with the multiplex RT-PCR (VL corr. simult.)) are shown in the FIGURE. The coefficient of variation (CV) increased in three of the four samples (cat 1, 3 and 4) after the implementation of the correction factor of a separate real-time RT-PCR (VL corr. sep.). In contrast, the CV of the viral load data of all four samples is decreased after correction using the multiplex approach (VL corr. simult.).

### Influence of Mismatches on Reaction Efficiencies

Another factor, beside quantification and sample preparation, which could influence the viral load estimation is the presence of mismatches in the primer and probe binding region. This is of special interest when viral load data obtained from different isolates, e.g. in vaccination studies with different challenge viruses, should be compared. To be able to compare the viral load data, the difference in the reaction efficiencies of the corresponding real-time RT-PCRs must be negligible or must be compensated for. The reaction efficiency can be estimated from the slope of the standard curve. A comparison of the slopes of different standard curves is only possible if certain criteria are fulfilled. We chose the following criteria to give us the confidence to compare the reaction efficiencies: (i) each point of the standard curve is measured in triplicate (ii) the standard curve should contain at least 5 points distributed over at least 2 logarithmic decades (iii) the C_{T} values should be below 35 (iv) the coefficient of correlation (r²) of the 15 values (3×5 points) of the standard curve should be high (>0,993) (v) the batch of components used for the RT-PCR should be identical.

We harvested the supernatant of three CrFK cell lines persistently infected with different FIV isolates (Petaluma, Utrecht 113, Glasgow 8). After RNA extraction and fourfold serial dilution of the RNA we measured the dilution series in triplicate in seven real-time RT-PCR assays. The CVs together with the calculated reaction efficiencies of the resulting standard curves obtained for the real-time RT-PCRs of measured FIV isolates are shown in Table 4, section A.

In two cases (Petaluma in assay e and assay g) the CV values did not meet the above described criteria (CV>0.993) and thus we did not calculate the reaction efficiency from the slope of the standard curve.

### One Mismatch (Table 4, Section A, Assays a and b)

In assay a Petaluma and Utrecht 113 have no mismatches in the primer or probe binding region and displayed nearly identical reaction efficiencies (0.9702 and 0.9548). In contrast, Glasgow 8, which has a point mutation in the reverse primer has a lower reaction efficiency of 0.8088. Independently of the absolute reaction efficiency (which are consistently higher in assay b compared to assay a) a reduction of reaction efficiency of a similar magnitude can be observed between primer-probe combinations with no mismatches compared to those with one mismatch in one primer (assay a cf. Petaluma with Glasgow 8; assay b cf. Glasgow 8 with Utrecht 113).

### Two Mismatches (Table 4, Section A, Assays b, c, d)

The reaction efficiency is further reduced in assay b for Petaluma (0.9738), which has both, a point mutation in the reverse primer and an additional point mutation in the probe. A similar reduction of the reaction efficiency (from 1.0783 to 0.9761 in assay c) between Glasgow 8 (without mutations) and Petalumna (with a point mutation in the reverse primer and the probe) is observed, if a different forward primer (FIV1366fa) is used in combination with the same reverse primer and probe (see Table 4a, assay c). A sub-optimal reverse primer with two point mutations in assay d further reduces the reaction efficiency (1.0026 from Glasgow 8 in assay d compared to 1.1106 in assay b).

### Three Mismatches (Table 4, Section A, Assay d)

An additional point mutation in the forward primer in Utrecht 113 has a similar effect on the reduction of the reaction efficiency as an additional mutation in the probe in Petaluma.

### Position of Mismatch (Table 4, Section A, Assays d and e)

If a better forward primer (assay e, FIV1366fa with a point mutation at the 5′-end of Utrecht 113 compared to assay d, primer FIV1360f with a point mutation in the center) is used in combination with the sub-optimal reverse primer FIV1437rb, the reduction of the reaction efficiency compared to the Glasgow 8 isolate with only two mutations is decreased.

Although some general trends are apparent in these experiments (i.e. mismatches result in a reduction of reaction efficiency), the number of mismates, their location and the type of the resulting mismatch have profound effect upon the absolute reduction in reaction efficiency. We were interested to see to what extent these differences in reaction efficiency translate into viral load measurements.

### Impact of Mismatches on Viral Load.

In order to determine this the standard curves obtained for the same samples of one isolate (Petaluma) with two different real-time RT-PCR assays (Table 4, section B: assay f without mutations and assy g with two mutations in the reverse primer) were compared. Surprisingly, the two mutations resulted in differences of measured C_{T} values of between 11 and 13.5 dependent on the amount of RNA template used for the assay. Such differences are equivalent to differences in viral load between 3.3 and 4.2 logarithmic decades.

Table 1: Comparison of PCR-efficiencies of the FIV1010p-assay and sequence variation in the oligonucleotide binding site.

The PCR-efficiencies of four different FIV isolates, and the corresponding sequences of the PCR-products are listed. The sequence given in this table is always from the same strand, despite the fact that the probe and the reverse primer bind to the complementary strand compared to the forward primer. The exact sequences (5′- 3′orientation) of the primers and probe are described above.

TABLE 1

| | | |
|---|---|---|
| Origin of the | PCR- | Sequence |

| | | | | |
|---|---|---|---|---|
| sequence | efficiency | FIV0771f | FIV1010p | FIV1081r |

| | | | | |
|---|---|---|---|---|
| Oligonucl. | | agaacctggtgatataccagagac | aggagggccctccacaggcata | caatatgtagcacttgacccaa |
| | | (SEQ ID NO.:1) | | |
| Petaluma | 0.9742 | ------------------------ | ---------------------- | ---------------------- |
| Glasgow8 | 0.9284 | ------------------------ | ---------------------- | -----------t---------- |
| Amsterdam6 | 0.8711 | ------------------------ | ----------c----------- | ---------------------- |
| Utrecht113 | 0.9485 | ------------------------ | ---------------------- | ---------------------- |

Table 2: Comparison of the real-time PCR results and sequence variation in the probe-binding site.

A comparison of the results from the real-time PCR and the sequence of the probe-binding site of PCR-products derived from four field isolates. The nucleotide sequence in the table is complementary to the sequence of the probe used. (nd) not determined.

TABLE 2

| | Agarose | | |
|---|---|---|---|
| Field isolate | electrophoresis | gel Real-Time PCR | FIV101P binding site |
| Oligonucl. | | | agg agg gcc ctc cac agg cat a |
| Munich 3 | + | − | -a- --- -a- --- --- --- -t- - |
| Munich 4 | + | − | -g- --a aa- --- --- --- --- - |
| Munich 5 | − | − | nd |
| Munich 6 | + | + | --- --- --- --- --- --- --- - |
| Munich 7 | + | + | --- --- --- --- --- --- --- - |
| Munich 1 | + | + | nd |
| Munich 2 | + | + | nd |
| Munich 8 | − | − | nd |
| Munich 9 | + | + | nd |
| Munich 10 | − | − | nd |

Table 3: Amplification of 25 unknown FIV isolates by three monoplex or two multiplex real-time PCRs.

Results of the amplification of 30 unknown FIV isolates by the two monoplex real-time PCR assays 1010p and 1372p or by one multiplex real-time PCR composed of the 1010v- and the 1372p-assay. The subtype of the isolate was identified according to the results of the real-time PCR: (+) a PCR-product was detected with this assay; (−) no PCR-product was detected using this assay; (*) the subtype can only be determined by a multiplex real-time PCR.

TABLE 3

PCR-assay

| FIV isolate | 1010p | 1372p | 1010v/1372p | subtype |
|---|---|---|---|---|
| Munich 11 | + | − | +/− | A |
| Munich 14 | − | + | −/+ | B |
| Munich 18 | − | + | −/+ | B |
| Munich 20 | − | + | −/+ | B |
| Munich 27 | − | + | −/+ | B |
| Munich 29 | − | + | −/+ | B |
| Munich 31 | + | − | +/− | A |
| Munich 32 | + | − | +/− | A |
| Munich 35 | + | − | +/− | A |
| Munich 36 | + | − | +/− | A |
| Munich 38 | − | + | −/+ | B |
| Munich 39 | − | + | −/+ | B |
| Munich 40 | − | + | −/+ | B |
| Munich 41 | + | − | +/− | A |
| Munich 43 | − | + | −/+ | B |
| Munich 44 | + | − | +/− | A |
| Munich 49 | − | + | −/+ | B |
| Munich 50 | − | + | −/+ | B |
| Munich 52 | + | − | +/− | A |
| Munich 53 | + | − | +/− | A |
| Utrecht 113 | + | − | +/− | A |
| Petaluma | + | − | +/− | A |
| Amsterdam 6 | + | − | +/− | A |
| Italy M2* | + | + | −/+ | B |
| Italy M20* | + | + | −/+ | B |
| Austria 01* | + | + | −/+ | B |
| Austria 02 | + | − | +/− | A |
| Austria TE* | + | + | −/+ | B |
| Austria 05* | + | + | −/+ | B |
| Austria 06* | + | + | −/+ | B |

Table 4: The coefficients of correlation of the standard curves and the calculated reaction efficiencies of the three FIV isolates.

The coefficients of correlation of the standard curves and the calculated reaction efficiencies of the three FIV isolates are illustrated together with mismatches found in the primer or probe binding region of the seven real-time RT-PCR assays used (assays a-g). The sequence given in these tables is always from the same strand (positive DNA strand), despite the fact that the reverse primer binds to the complementary strand. The exact sequences (5′- 3′ orientation) of the primers and probe are described above. A). Assays a-e were used to determine the influence of mismatches on the reaction efficiency. B). Assays f and g were necessary to demonstrate the influence of mismatches in the primer binding region on viral load measurements. C). In order to illustrate the type of the resulting mismatch in binding region of primer FIV1437rb (assay d and e) and primer FIV 1307r (assay g) the positive DNA strand of Glasgow 8 (assays d and e) together with primer FIV1437rb as well as the positive DNA strand of Petaluma (assay e) together with primer FIV1307r is shown.

TABLE 4

| | | | | | | |
|---|---|---|---|---|---|---|
| | | coeffi- | | | | |
| | | cient | | | | |
| | | of | reaction | | | |
| As- | FIV | corre- | effi- | | | |
| say | Isolate | lation | ciency | forward primer | probe | reverse primer |

| | | | | | | |
|---|---|---|---|---|---|---|
| A | | | | FIV0771f | FIV1010p | FIV1081r |
| a | | | | | | |
| | | | | AGA ACG TGG TGA | AGG AAG GCC CTC | CAA TAT GTA GCA |
| | | | | TAT ACC AGA GAC | CAC AGG CAT A | CTT GAC CCA A |
| | | | | (SEQ ID NO.:1) | | |
| | Petaluma | 0.9995 | 0.9702 | --- --- --- --- --- --- | --- --- --- --- --- --- | --- --- --- --- --- --- --- - |
| | | | | --- --- | --- - | |
| | Utrecht | 0.9985 | 0.9548 | --- --- --- --- --- --- | --- --- --- --- --- --- | --- --- --- --- --- --- --- - |
| | 113 | | | --- --- | --- - | |
| | Glasgow | 0.9969 | 0.8088 | --- --- --- --- --- --- | --- --- --- --- --- --- | --- --- --- --T --- --- --- - |
| | 8 | | | --- --- | --- - | |
| b | | | | FIV136Of | FIV1416p | FIV1437r |
| | | | | GCA GAA GCA AGA | TGC AGT GTA GAG | AGG AAA ATT GGG |
| | | | | TTT GCA CCA | CAT GGT ATG TTG | CGC CAT A |
| | | | | (SEQ ID NO.:4) | AGG CA | |
| | Glasgow | 0.9979 | 1.1106 | --- --- --- --- --- --- | --- --- --- --- --- --- | --- --- --- --- --- --- - |
| | 8 | | | --- | --- --- --- -- | |
| | Utrecht | 0.9944 | 1.0342 | --- --- C-- --- --- --- | --- --- --- --- --- --- | --- --- --- --- --- --- - |
| | 113 | | | --- | --- --- --- -- | |
| | Petaluma | 0.9938 | 0.9738 | --- --- --- --- --- --- | --- --- --- --- --- --- | --- --- --- --- T-- --- - |
| | | | | --- | --- -C- --- -- | |
| c | | | | FIV1366fa | FIV1416p | FIV1437r |
| | | | | GCA AGA TTT GCA | TGC AGT GTA GAG | AGG AAA ATT GGC |
| | | | | CCA GCT AGG | CAT GGT ATC TTG | CGC CAT A |
| | | | | (SEQ ID NO.:14) | AGG CA | |
| | Glasgow | 0.9985 | 1.0783 | --- --- --- --- --- --- | --- --- --- --- --- --- | --- --- --- --- --- --- - |
| | 8 | | | --- | --- --- --- -- | |
| | Petaluma | 0.9971 | 0.9761 | --- --- --- --- --- --- | --- --- --- --- --- --- | --- --- --- --- T-- --- - |
| | | | | --- | --- -C- --- -- | |
| d | | | | FIV1360f | FIV1416p | FIV1437rb |
| | | | | GCA GAA GCA AGA | TGC AGT GTA GAG | AGG AAA GTT GGC |
| | | | | TTT GCA CCA | CAT GGT ATC TTG | AGC CAT A |
| | | | | (SEQ ID NO.:4) | AGG CA | |
| | Glasgow | 0.9986 | 1.0026 | --- --- --- --- --- --- | --- --- --- --- --- --- | --- --- A-- --- C-- --- - |
| | 8 | | | --- | --- --- --- -- | |
| | Utrecht | 0.9978 | 0.9400 | --- --- C-- --- --- --- | --- --- --- --- --- --- | --- --- A-- --- C-- --- - |
| | 113 | | | --- | --- --- --- -- | |
| | Petaluma | 0.9984 | 0.9440 | --- --- --- --- --- --- | --- --- --- --- --- --- | --- --- A-- --- T-- --- - |
| | | | | --- | --- -C- --- -- | |
| e | | | | FIV1366fa | FIV1416p | FIV1437rb |
| | | | | GCA AGA TTT GCA | TGC AGT GTA GAG | AGG AAA GTT GGC |
| | | | | CCA GCT AGG | CAT GGT ATC TTG | AGC CAT A |
| | | | | (SEQ ID NO.:14) | AGG CA | |
| | Glasgow | 0.9983 | 1.0385 | --- --- --- --- --- --- | --- --- --- --- --- --- | --- --- A-- --- C-- --- - |
| | 8 | | | --- | --- --- --- -- | |
| | Utrecht | 0.9982 | 0.9994 | C-- --- --- --- --- --- | --- --- --- --- --- --- | --- --- A-- --- C-- --- - |
| | 113 | | | --- | --- --- --- -- | |
| | Petaluma | 0.9261 | nc | --- --- --- --- --- --- | --- --- --- --- --- --- | --- --- A-- --- T-- --- - |
| | | | | --- | --- -C- --- -- | |
| B | | | | FIV1182f | FIV1212v | FIV13O7z |
| f | | | | | | |
| | | | | ATG GCC ACA TTA | TGC GCT GCA GAT | TGA TGC TCC CAG |
| | | | | ATA ATG GC | AAA GAA ATA TTG | ACC ATTA CC |
| | | | | (SEQ ID NO.:16) | GAT GA | |
| | Petaluma | 0.9941 | 1.0980 | --- --- --- --- --- --- | --- --- --- --- --- --- | --- --- --- --- --- --- --- |
| | | | | -- | --- --- --- -- | |
| g | | | | FIV1182f | FIV1212v | FIV13O7r |
| | | | | ATG GCC ACA TTA | TGC GCT GCA GAT | TGA TGG TCC TAG |
| | | | | ATA ATG GC | AAA GAA ATA TTG | ACC ATT ACC |
| | | | | (SEQ ID NO.:16) | GAT GA | |
| | Petaluma | 0.9609 | nc | --- --- --- --- --- --- | --- --- --- --- --- --- | --- --C --- C-- --- --- --- |
| | | | | -- | --- --- --- -- | |
| C | | | | | | FIV1437rb |
| | | | | | positive DNA strand | AGG AAA ATT GGC |
| | | | | | (Glasgow 8) | CGC CAT A |
| | | | | | reverse primer | TCC TTT CAA CCG |
| | | | | | | TCG GTA T |
| | | | | | | FIV1307r |
| | | | | | positive DNA strand | TGA TGC TCC CAG |
| | | | | | (Petaluma) | ACC ATT ACC |
| | | | | | reverse primer | ACT ACC AGG ATC |
| | | | | | | TGG TAA TGG |

While this invention has been particularly shown and described with references to preferred embodiments thereof, it will be understood by those skilled in the art that various changes in form and details may be made therein without departing from the scope of the invention encompassed by the appended claims.

What is claimed is:

1. A real-time Polymerase Chain Reaction (PCR) method for the detection and/or quantification of variants of a nucleic acid sequence, wherein the same region of said variants is completely and/or partially to be amplified, each variant differing in one or more nucleotides within a probe-binding site, said method comprising adding two or more oligonucleotides probes to the same PCR mixture, each probe being specific for the probe binding site of at least one variant.

2. The real-time PCR method according to claim 1, wherein said variants of the nucleic acid sequence differ in one or more nucleotides within the primer binding sites and wherein more than one primer pair is added to the reaction mixture each primer specifically annealing to the primer binding site of at least one subtype.

3. The real-time PCR method according to claim 1, wherein two or more parts of the region are amplified, each part of the region comprising only one probe binding site.

4. The real-time PCR method according to claim 1, wherein the probes are labeled with different fluorescent reporter dyes.

5. The real-time PCR method according to claim 4, wherein the probes are labeled with FAM™ or VIC™.

6. The real-time PCR method according to claim 1 wherein the nucleic acid sequence is a viral nucleic acid sequence.

7. The real-time PCR method according to claim 6 wherein the viral nucleic acid sequence is a retroviral nucleic acid sequence.

8. The real-time PCR method according to claim 7 wherein the retroviral nucleic acid sequence is a lentiviral nucleic acid sequence.

9. The real-time PCR method according to claim 8 wherein the lentiviral nucleic acid sequence is a Feline Immunodeficiency Viral (FIV) nucleic acid sequence.

10. The real-time PCR method according to claim 9, wherein the probes comprise SEQ ID NO.:3 and SEQ ID NO.:6 or SEQ ID NO.:24.

11. The real-time PCR method according to claim 9, wherein the probes comprise SEQ ID NO.:3 and SEQ ID NO.:9 or SEQ ID NO.:24.

12. The real-time PCR method according to claim 9, wherein a forward primer and a reverse primer are added to the mixture, and the forward primer is selected from the group consisting of; SEQ ID NO.:1, SEQ ID NO.:12, SEQ ID NO.:22 and combinations thereof, and the reverse primer is selected from the group consisting of; SEQ ID NO.:2, SEQ ID NO.:23 and combinations thereof.

13. The real-time PCR method according to claim 9, wherein a forward primer and a reverse primer are added to the mixture, and the forward primer is selected from the group consisting of: SEQ ID NO.:4, SEQ ID NO.:14, SEQ ID NO.:15 and combinations thereof, and the reverse primer is selected from the group consisting of: SEQ ID NO.:5, SEQ ID NO.:13 and combinations thereof.

14. The real-time PCR method according to claim 9, wherein a forward primer and a reverse primer are added to the mixture, and the forward primer is selected from the group consisting of: SEQ ID NO.:7, SEQ ID NO.:20, SEQ ID NO.:21 and combinations thereof, and the reverse primer is SEQ ID NO.:8.

15. The real-time PCR method according to claim 9, wherein a forward primer and a reverse primer are added to the mixture, and the forward primer is SEQ ID NO.:16, and the reverse primer is selected from the group consisting of: SEQ ID NO.:17, SEQ ID NO.:19 and combinations thereof.

16. The real-time PCR method according to claim 9, wherein forward primers and reverse primers are added to the reaction mixture, and the forward primers are selected from the group consisting of: SEQ ID NO.:1, SEQ ID NO.:4, SEQ ID NO.:12, SEQ ID NO.:14, SEQ ID NO.:15, SEQ ID NO.:22 and combinations thereof; the reverse primers are selected from the group consisting of: SEQ ID NO.: 2, SEQ ID NO.:5, SEQ ID NO.:13, SEQ ID NO.:23 and combinations thereof; and the probes are selected from the group consisting of: SEQ ID NO.:3, SEQ ID NO.:6, SEQ ID NO.:24 and combinations thereof.

17. The real-time PCR method according to claim 9, wherein forward primers and reverse primers are added to the mixture, and the forward primers are selected from the group consisting of: SEQ ID NO.:1, SEQ ID NO.:7, SEQ ID NO.:12, SEQ ID NO.:20, SEQ ID NO.:21, SEQ ID NO.:22, and the combinations thereof; the reverse primers are selected from the group consisting of: SEQ ID NO.:2, SEQ ID NO.:8, SEQ ID NO.:23 and combinations thereof; and the probes are selected from the group consisting of: SEQ ID NO.:3, SEQ ID NO.:9, SEQ ID NO.:24 and combinations thereof.

18. The real-time PCR method according claim 1, wherein said PCR is a reverse-transcription (RT) PCR.

19. The real-time PCR method according to claim 1, wherein said variants of nucleic acid sequences are nucleic acid sequences derived from subtypes, isolates, clades or any other subgroup of a species.

20. The real-time PCR method according to claim 1, wherein in the same one-tube reaction a standard nucleic acid sequence is simultaneously amplified and quantified according to real-time PCR principles.

21. The real-time PCR method according to claim 20, wherein the standard nucleic acid sequence is part of a cellular genome.

22. The real-time PCR method according to claim 20, wherein the standard nucleic acid sequence is added in a known copy number to a sample to be tested.

23. The real-time PCR method according to claim 21, wherein the standard nucleic acid sequence derives from the nucleic acid sequence encoding the EGPF (green fluorescence) gene or the 18S rDNA gene.

24. A real-time Polymerase Chain Reaction (PCR) method for the determination of the overall viral load in a sample comprising variants of a viral nucleic acid sequence comprising adding two or more oligonucleotide probes to a PCR mixture, each probe being specific for a probe binding site of at least one of the variants.

25. The method according to claim 24, wherein the variants are derived from nucleic acid sequences derived from subtypes, isolates, clades or any other subgroup of a viral species.

26. A real-time Polymerase Chain Reacton (PCR) method for the determination of the impact of the viral load on tumorgenesis comprising adding two or more oligonucleotide probes to a PCR mixture, each probe being specific for a probe binding site of at lease one viral variant.

27. A real-time Polymerase Chain Reaction (PCR) method for the determination of nucleic acid extraction efficiency or transfection efficiency comprising adding two or more oligonucleotide probes to a PCR mixture, each probe being specific for a probe binding site of a variant.

28. An oligonucleotide probe selected from the group consisting of: SEQ ID NO.:6, SEQ ID NO.:9, SEQ ID NO.:18, SEQ ID NO.:24 and complementary strands thereof.

29. A primer selected from the group consisting of: SEQ ID NO.:4, SEQ ID NO.:5, SEQ ID NO.:7, SEQ ID NO.:8, SEQ ID NO.:10, SEQ ID NO.:17, SEQ ID NO.: 19, SEQ ID NO.:20, SEQ ID NO.:21, SEQ ID NO.:22, and SEQ ID NO.:23.

30. A set of primers selected from the group consisting of SEQ ID NO.:2 and SEQ ID NO.:12.

31. A set of primers selected from the group consisting of: SEQ ID NO.:4, SEQ ID NO.:5, SEQ ID NO.:13, SEQ ID NO.:14, SEQ ID NO.:15 and combinations thereof.

32. A set of primers selected from the group consisting of: SEQ ID NO.:7, SEQ ID NO.:8, SEQ ID NO.:20, SEQ ID NO.:21 and combinations thereof.

33. A set of primers selected from the group consisting of: SEQ ID NO.:16, SEQ ID NO.:17, SEQ ID NO.:19 and combinations thereof.

34. A set of primers selected from the group consisting of: SEQ ID NO.:10, SEQ ID NO.:22, SEQ ID NO.:23 and combinations thereof.

35. A set of oligonucleotides comprising a primer set selected from the group consisting of SEQ ID NO.:2, SEQ ID NO.:4, SEQ ID NO.:5, SEQ ID NO.:7, SEQ ID NO.: 8, SEQ ID NO.:10, SEQ ID NO.:12, SEQ ID NO.:13, SEQ ID NO.:14, SEQ ID NO.:15,SEQ ID NO.:16,SEQ ID NO.:17,SEQ ID NO.:19,SEQ ID NO.:20,
SEQ ID NO.:21, SEQ ID NO.:22, SEQ ID NO.:23 and combinations thereof, and a probe selected from the group consisting of: SEQ ID NO.:6, SEQ ID NO.:9, SEQ ID NO.:18, SEQ ID NO.:24 and complementary strands thereof.
